(19) United States
(12) Patent Application Publication
(10) Pub. No.: US 2019/0016344 A1
(43) Pub. Date: Jan. 17, 2019

(54) METHOD FOR ADJUSTING AT LEAST ONE OPERATING PARAMETER OF A TRANSPORTATION VEHICLE, SYSTEM FOR ADJUSTING AT LEAST ONE OPERATING PARAMETER OF A TRANSPORTATION VEHICLE, AND TRANSPORTATION VEHICLE

(71) Applicant: VOLKSWAGEN AG, Wolfsburg (DE)

(72) Inventor: Sebastian EHMANN, Wolfenbüttel (DE)

(21) Appl. No.: 16/029,551

(22) Filed: Jul. 7, 2018

(30) Foreign Application Priority Data
Jul. 12, 2017 (DE) 10 2017 211 931.4

(51) Int. Cl. B60W 50/08; B60W 50/00; A61B 5/18; A61B 5/0205; A61B 5/0476

(52) U.S. Cl. CPC B60W 50/085; A61B 5/024; A61B 5/18; A61B 5/0205; A61B 5/0476; B60W 2540/02; B60W 2540/10; B60W 2540/28; B60W 2540/12; B60W 2540/18; B60W 2540/22; B60W 2540/26; B60W 2520/105; B60W 2520/125; B60W 2550/14; B60W 50/0098

(57) ABSTRACT

A method for adapting at least one operating parameter of a transportation vehicle during a driving maneuver of the transportation vehicle, wherein the at least one operating parameter of the transportation vehicle is set by at least one driver assistance system of the transportation vehicle, wherein the method detects at least one activity by a transportation vehicle occupant during the driving maneuver in response to the driving maneuver by a detection device and adapts the at least one operating parameter of the transportation vehicle in response to the detected activity by the transportation vehicle occupant by activating the at least one driver assistance system using a control system.

### PRIORITY CLAIM

This patent application claims priority to German Patent Application No. 10 2017 211 931.4, filed 12 Jul. 2017, the disclosure of which is incorporated herein by reference in its entirety.

### SUMMARY

Illustrative embodiments relate to a method for adapting at least one operating parameter of a transportation vehicle. Illustrative embodiments also relate to a system for adapting at least one operating parameter of a transportation vehicle as well as a transportation vehicle having a system of this type.

### BRIEF DESCRIPTION OF THE DRAWINGS

Exemplary embodiments will be described below with reference to the drawing, in which:

FIG. 1 shows a schematic view of the disclosed method for adapting at least one operating parameter of a disclosed transportation vehicle in a flow chart.

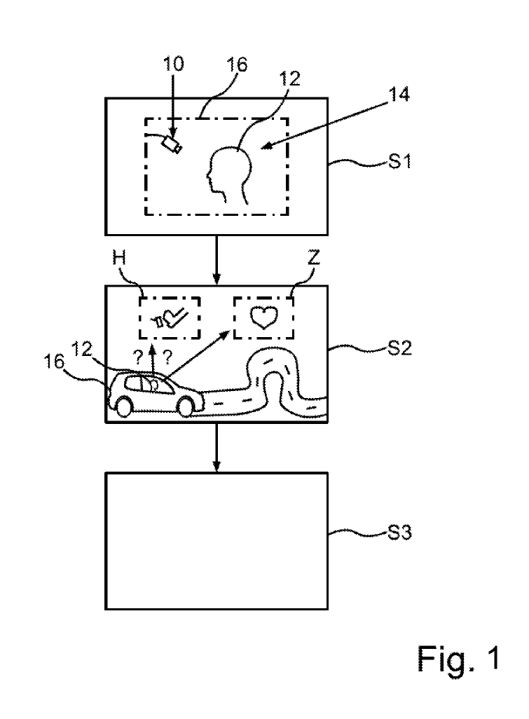

### DETAILED DESCRIPTION

The characteristics and design of driver assistance functions, which are controlled by one or more driver assistance systems of a transportation vehicle, depend significantly upon the subjective perception of the expected passengers, i.e., the transportation vehicle occupants, in the transportation vehicle. For example, when the driver assistance system is an adaptive cruise control, which regulates the velocity of the transportation vehicle and takes into account whether other transportation vehicles are located in front of the subject transportation vehicle, it is possible to set the minimum distance from the next transportation vehicle at which a braking procedure is initiated. In this case, it depends upon the perception of the respective transportation vehicle occupants whether the braking procedure commences too soon or too late or whether the deceleration is perceived as too weak or too strong. A further example is lateral acceleration, which occurs while driving through a curve with a driver assistance system, such as a lane departure warning system. A lateral acceleration and the associated transit speed through the curve that is still acceptable to the transportation vehicle occupant may already be considered unpleasant by another transportation vehicle occupant.

It is known from the general prior art that, when a driver assistance system is used, a parameter can be adjusted by the transportation vehicle occupant, such as the driver, or can be programmed by the transportation vehicle occupant. In this regard, DE 10 2014 118 079 A1, for example, discloses a transportation vehicle having an autonomous driving sensor that is configured to detect at least one state while the transportation vehicle is operated in an autonomous mode. A processing system in the transportation vehicle is designed to control at least one transportation vehicle sub-system while the transportation vehicle is operated in the autonomous mode. Moreover, the processing system is designed to control at least one transportation vehicle sub-system in accordance with a driver preference.

Rather than defining parameters for a driver assistance system, the transportation vehicle itself can be configured to act proactively. To this end, EP 2 862 773 A2 describes a transportation vehicle comprising a driver assistance system for performing advanced calculations of forecast data about at least one future driving situation of the transportation vehicle by evaluating the ego data relating to the transportation vehicle and the environmental data relating to the transportation vehicle environment. In a first operating mode of the driver assistance system, the transportation vehicle can be controlled by a driver, wherein, when a trigger condition is satisfied, the driver assistance system is configured to switch temporarily into a second operating mode in which the control of the transportation vehicle is carried out autonomously by the driver assistance system without the possibility of intervention by the driver. The trigger condition is designed to evaluate at least the forecast data and the driver characteristic data describing at least one driver characteristic.

The drawback of driver assistance systems of this type is that, if the driver or passenger of the transportation vehicle should feel uneasy as a result of the pre-set parameters, the transportation vehicle must either be operated in a manual driving mode or the parameters must be further adjusted by the driver himself or herself. In an increasing number of driver assistance systems, it has become more and more laborious for the transportation vehicle occupant to define all of the parameters himself or herself. Parameter values that are acceptable for the transportation vehicle occupant can also fluctuate throughout the day or depending upon a personal condition of the transportation vehicle occupant. Likewise, the parameter ranges can fluctuate as a function of the number and type of transportation vehicle occupants.

Therefore, disclosed embodiments provide a method for adapting at least one operating parameter of a transportation vehicle as well as a system for adapting at least one operating parameter of the transportation vehicle, in which the at least one operating parameter can be adapted in an especially simple way and reliability is thereby increased, in particular, with regard to a compatibility of the adapted operating parameter for a driver or passenger of the transportation vehicle.

Disclosed embodiments provide a method for adapting at least one operating parameter of a transportation vehicle during a driving maneuver of the transportation vehicle, wherein the at least one operating parameter of the transportation vehicle is set by at least one driver assistance system of the transportation vehicle. The term “operating parameter” means a parameter that describes a driving profile or a driving status or a driving property of the transportation vehicle, i.e., a driving parameter. Optionally, a velocity of the transportation vehicle and/or an acceleration, especially a lateral acceleration, of the transportation vehicle and/or a longitudinal acceleration of the transportation vehicle and/or a distance from a transportation vehicle driving ahead and/or the keeping of the transportation vehicle in a driving lane and/or a steering torque is adapted as the at least one operating parameter. The phrase “adapting the at least one operating parameter” is intended to mean a modification or setting or switching of a predetermined or pre-set operating parameter to a new operating parameter. “Driving maneuver” refers to an action or the performance of a maneuver by the transportation vehicle. For example, the driving maneuver can be the driving of the transportation vehicle and/or passing another transportation vehicle and/or changing lanes and/or turning a corner and/or driving backward and/or stopping and/or starting and/or accelerating and/or driving through curves and/or pulling into a parking space and/or pulling out of a parking space. The set operating parameter defines or influences the driving maneuver. For example, a driver assistance system can be configured to monitor and/or control the negotiation of a curve by the transportation vehicle. To this end, a lateral acceleration can be specified to the driver assistance system as an operating parameter for carrying out the driving through the curve as a driving maneuver.

In at least one operation of the method, at least one activity by a transportation vehicle occupant during the driving maneuver in reaction to the driving maneuver is detected by a detection device. Here, the detection device can be configured to detect a current driving maneuver of the transportation vehicle. Alternatively, the at least one driver assistance system can signal to the driver, whereupon a control system can activate the detection device. During the detected driving maneuver, the transportation vehicle occupant may be monitored or observed. In other words, the detection device can be designed to detect a change in a behavior and/or state of the transportation vehicle occupant during the driving maneuver. The transportation vehicle occupant can be a driver or a passenger of the transportation vehicle, for example. Alternatively or additionally, the detection device can be configured to detect at least one further transportation vehicle occupant and to detect at least one activity by the further transportation vehicle occupant during the driving maneuver in reaction to the driving maneuver by the detection device.

In a further method operation, the at least one operating parameter of the transportation vehicle is adapted or set in response to the detected activity by the transportation vehicle occupant by activating the at least one driver assistance system by a control system. To do so, it is possible to modify a value of the at least one operating parameter of the transportation vehicle which influences the current driving maneuver or with which the current driving maneuver is carried out. In addition, it is possible that the at least one operating parameter is adapted depending upon the detected activity of the transportation vehicle occupant and depending upon the current driving maneuver. In adapting the at least one operating parameter, the control system can be configured to increase or reduce a value of the operating parameter. For the control system to be able to reliably adapt the at least one operating parameter to the reaction of the at least one transportation vehicle occupant, multiple activities can be saved in a memory device of the control system. Depending upon which activity is detected, the control system can be configured to increase or decrease the currently set value of the at least one operating parameter, in other words, to turn it up or turn it down. If no activity or no change in the activity of the at least one transportation vehicle occupant is detected by the detection device, then the at least one operating parameter may not be adapted. In other words, if no activity or no change in the activity of the at least one transportation vehicle occupant is detected by the detection device, then the currently set operating parameter is maintained.

Multiple operating parameters can also be associated with a driving maneuver. For example, when the driving maneuver is negotiating a curve, a steering torque and a lateral acceleration can be specified. Accordingly, multiple operating parameters can also additionally or alternatively be adapted by the control system as a function of the driving maneuver.

Using the method, a direct link is established between the operating parameter and a driving operation of the transportation vehicle. In so doing, the at least one operating parameter can be adapted in direct reaction to the activity of the transportation vehicle occupant, since the reaction was brought about by the driving maneuver. The needs or the behavior or the activity of the transportation vehicle occupant can be addressed directly, especially fast and reliably by this method.

At least one disclosed embodiment provides that the heart rate and/or the pulse and/or the respiratory rate and/or the brain activity and/or the drowsiness of the transportation vehicle occupant is detected as the activity of the transportation vehicle occupant. In other words, a state parameter of the transportation vehicle occupant is recorded. To detect the transportation vehicle occupant's heart rate, the detection device can comprise a heart rate monitor and/or a fitness wristband, for example. To detect the transportation vehicle occupant's respiratory rate and/or the transportation vehicle occupant's drowsiness, the detection device can include a camera, for example. For example, based on image data from the camera, the frequency with which the transportation vehicle occupant's chest rises and falls, i.e., how often this occurs, can be analyzed to determine the respiratory rate. Additionally or alternatively, a blinking frequency by the eyes of the transportation vehicle occupant can be detected based on the image data from the camera to ascertain the drowsiness. For instance, it is possible to detect whether or not the transportation vehicle occupant is sleeping as a state of drowsiness. To detect the transportation vehicle occupant's brain activity, the detection device can comprise an electroencephalogram (EEG), for example. Using these types of devices to detect the state parameter, a conclusion can be drawn about the activity of the transportation vehicle occupant in an especially simple and reliable way. If, for example, the transportation vehicle occupant's pulse is detected while the transportation vehicle is driving through a curve, and the pulse is above a predetermined limit or threshold value, then the control system can be configured to activate the driver assistance system in such a way that lateral acceleration is reduced. If, for example, the transportation vehicle occupant's pulse is detected during a braking procedure, and the pulse is above the predetermined threshold value, then the control system can be configured to activate the driver assistance system in such a way that the distance from a transportation vehicle driving ahead is increased by the initiation of a braking procedure.

Additionally or alternatively, a predetermined action by the transportation vehicle occupant that indicates an intervention by the transportation vehicle occupant in the driving maneuver is detected as the activity in a beneficial way. In other words, a movement of the transportation vehicle occupant with which the transportation vehicle occupant would like to intervene in the driving maneuver can be detected as the activity in question. Here, a foot movement by the transportation vehicle occupant that indicates an actuation of a brake pedal or an accelerator pedal of the transportation vehicle can be detected as the predetermined action. Alternatively or additionally, the predetermined action can indicate an actuation of a steering wheel of the transportation vehicle. For example, the detection device can have a camera and/or a sensor to detect steering wheel movements, and it or they can be configured to detect an approach by at least one of the transportation vehicle occupant's hands or contact with the steering wheel by at least one of the transportation vehicle occupant's hands. For example, the detection device can have a camera and/or a sensor to detect interventions on the brake pedal or accelerator pedal, and it or they can be configured to detect an approach by at least one of the transportation vehicle occupant's feet or contact with the respective pedal by the transportation vehicle occupant's foot.

Additionally or alternatively, a protective action by the transportation vehicle occupant for the personal protection of the transportation vehicle occupant can be detected as the activity by the transportation vehicle occupant. In other words, it is possible for the detection device to detect whether the transportation vehicle occupant has assumed a protective position in response to the driving maneuver as the activity by the transportation vehicle occupant. Optionally, a vocal utterance by the transportation vehicle occupant and/or holding tightly and/or bracing by the transportation vehicle occupant against an interior element of the transportation vehicle, in particular, a handgrip in an interior of the transportation vehicle and/or a seat back of a seat and/or an arm rest of the seat and/or a door of the transportation vehicle is detected as a protective action. A vocal utterance by the transportation vehicle occupant can be detected by the detection device, for example, as a statement or a command or a noise by the transportation vehicle occupant. To this end, the detection device can have a speech recognition system, such as a microphone. The transportation vehicle occupant can express or say phrases such as “that was close” or “don't tailgate” or “a little slower” as the statement or command. A sigh or sounds that indicate or suggest fear or a groan can be detected by the detection device as the noise. For example, acoustic data of the noises and/or phrases and/or statements and/or commands can be saved in a memory device of the detection device to detect the vocal utterances of the transportation vehicle occupant. A control system of the detection device can be configured to compare the detected vocal utterances with the saved acoustic data. For instance, if the detection device detects the word “slower”, the control system can be configured to reduce the current value of the at least one operating parameter. To detect that the transportation vehicle occupant is holding tightly to and/or bracing against an interior element, such as a seat or a hand grip of the transportation vehicle, the detection device can have, for example, a sensor, in particular, a capacitive sensor, which is arranged on the at least one interior element.

The detection device can thus be designed to analyze and evaluate or detect conscious and/or unconscious behavior of the transportation vehicle occupant in reaction to the current driving maneuver, i.e., how the transportation vehicle occupant reacts to the current driving maneuver. In this way, it can be determined in an especially reliable way which parameter ranges in which of the driver assistance systems are perceived or classified by the transportation vehicle occupant as pleasant and or unpleasant.

A disclosed embodiment provides that the transportation vehicle occupant is identified by the detection device before the activity by the transportation vehicle occupant is detected, wherein biometric features are detected during the identification of the transportation vehicle occupant. To this end, the transportation vehicle occupant can initially be detected by the detection device in the interior of the transportation vehicle before the detection of the at least one activity by the transportation vehicle occupant. For example, the body size of the transportation vehicle occupant and/or the face of the transportation vehicle occupant can be detected as biometric features. Additionally or alternatively, the weight of the transportation vehicle occupant and/or the voice of the transportation vehicle occupant can be detected to identify the transportation vehicle occupant. Additionally or alternatively, a child seat in the interior of the transportation vehicle can be detected by the detection device to identify the transportation vehicle occupant. For example, a predetermined driving profile can be saved, in which pre-set operating parameters of the driver assistance system are stored for the transportation vehicle occupant. Additionally or alternatively, following the detection or identification of the transportation vehicle occupant, a control system can be configured to communicate with a transportation vehicle-external unit for transmitting data, in particular, operating parameters, and to receive these data from the transportation vehicle-external unit. In this way, for example, parameter sets that define or describe a driving behavior of the identified transportation vehicle occupant can be transmitted to the transportation vehicle, in particular, to the at least one driver assistance system. For example, the parameter sets can define or describe a sporty driving behavior or a defensive driving behavior by the transportation vehicle occupant. If the transportation vehicle is started, then the pre-set parameter sets, in particular, the parameter sets assigned or allocated to the transportation vehicle occupant, or the at least one operating parameter can be pre-set. By detecting the at least one reaction of the transportation vehicle occupant to the driving maneuver, which is controlled or monitored by the at least one driver assistance system, these parameter sets for the transportation vehicle occupant and/or the at least one pre-set operating parameter can be adapted.

A disclosed embodiments provides that a transportation vehicle occupant who gets into a transportation vehicle for the first time and for whom no driving profile or no parameter set is saved in the transportation vehicle and/or no driving profile or no parameter set can be accessed from a transportation vehicle-external unit for transmitting data is identified as an unknown and/or new transportation vehicle occupant. A new driving profile is established for the unknown and/or new transportation vehicle occupant, wherein generally determined, pre-set operating parameters or parameter values are used as initial parameter values, or the unknown and/or new transportation vehicle occupant is asked to provide desired parameters. A sporty or defensive driving mode can be set as the driving mode, for example, and a corresponding parameter set is loaded for the initial parameter values. An approach such as this is beneficially when the unknown and/or new transportation vehicle occupant is the driver of the transportation vehicle.

Disclosed embodiments can provide that, once the detection device has detected all transportation vehicle occupants and it has been established that no driving profile and/or parameter set exists for this constellation of transportation vehicle occupants, a query is made as to whether a new driving profile should be saved for the driver and/or an existing driving profile of the driver should be supplemented by the new constellation of the transportation vehicle occupants. A query relating to the saving of a new driving profile for the driver and/or the supplementing of an existing driving profile offers the benefit that it is possible to react to changes in the constellation of transportation vehicle occupants.

The position of the transportation vehicle occupants in the transportation vehicle could also be taken into consideration. Whether a transportation vehicle occupant is located in the passenger seat or in a back seat could be considered. For example, a transportation vehicle occupant, especially a child, in a rear seat might become car sick more quickly than if the transportation vehicle occupant were seated in the passenger seat. Accordingly, if the transportation vehicle occupant, especially the child, were positioned in the back seat, a more defensive driving mode would have to be employed than if the transportation vehicle occupant, especially the child, were seated in the passenger seat.

A further disclosed embodiment provides that, to adapt the at least one operating parameter of the transportation vehicle, the system detects a confirmation action to a status query that is produced by an output device with respect to the reaction by the transportation vehicle occupant to the driving maneuver. For example, the output device can be a screen, in particular, for the written output of the status query, and/or a speaker, in particular, for the acoustic output of the status query. The output device may be disposed in the interior of the transportation vehicle. If the output device is a screen, for example, then it can be arranged in the driver's field of vision, for instance, on a center console or on an instrument panel of the transportation vehicle. The output device can be configured to inquire as to the condition of the transportation vehicle occupant by activating a control system. For example, the output device can be configured to produce a written and/or acoustic phrase, especially a question, relating to the state of the transportation vehicle occupant. For instance, the question “Did the transportation vehicle just merge in or out too close?” or “Did the transportation vehicle just pass through the curve quickly?” or “Is everything all right?” can be produced as the phrase. In response, the detection device can be configured to detect a confirmation action or an agreement action to the status query by the transportation vehicle occupant. For example, the detection device can be configured to detect nodding or head shaking by the transportation vehicle occupant or an operational action on an actuation element or a vocal utterance, such as “yes” or “no”. It can be ensured in this way that the operating parameters are not adapted inadvertently, that is, without the agreement of the transportation vehicle occupant. Additionally, based on his or her reaction, the transportation vehicle occupant receives a direct response, whereby the transportation vehicle occupant can determine that his or her reaction has been understood.

According to a disclosed embodiment, the detection device additionally detects data from a device that include appointment entries and/or personal calendar data, which are taken into consideration when the at least one operating parameter is adapted. The “device” may be a transportation vehicle-external unit, in particular, an Internet server device or a portable mobile terminal. In other words, further data or data sets can be taken into consideration during the adaptation of the at least one operating parameter. The further data can be, for example, data from an appointment calendar that is saved on a portable mobile terminal coupled with the transportation vehicle. For instance, the detection device can recognize by a comparison or detection of further data whether the transportation vehicle occupant must arrive at an appointment quickly and thus expects a quicker driving mode.

The transportation vehicle is operated manually or partially autonomously or autonomously, especially in a fully automated way, during the driving maneuver. In this instance, “autonomous” means that the transportation vehicle moves independently. In the autonomous driving mode, it is possible that nobody is in the transportation vehicle or that no active driver or only passengers are in the transportation vehicle. For example, the transportation vehicle is controlled by the at least one driver assistance system or a plurality of driver assistance systems when in autonomous driving mode. In the autonomous driving mode, the driver of the transportation vehicle no longer has to handle the further operation of the transportation vehicle. For example, the transportation vehicle can drive through a curve autonomously. In this context, the term “manual” means that the transportation vehicle is controlled by a driver. “Partially autonomous” means that the transportation vehicle is operated autonomously for pre-determined driving maneuvers, such as parking, and is controlled by the driver in a driving mode.

Disclosed embodiments also include a system for adapting at least one operating parameter of the transportation vehicle during a driving maneuver of the transportation vehicle. The system comprises at least one driver assistance system, which is configured to set the at least one operating parameter. Moreover, the system has a detection device which is configured to detect at least one activity by a transportation vehicle occupant during the driving maneuver in reaction to the driving maneuver. Finally, the system has a control system which is configured to adapt the at least one operating parameter of the transportation vehicle in response to the detected activity by the transportation vehicle occupant by activating the at least one driver assistance system. The driver assistance system can be, for example, an adaptive cruise control and/or a lane departure warning system and/or a lane-change assistance system and/or a parking assistance system and/or a an emergency braking assistance system and/or an intelligent speed assistance system and/or an acceleration assistance system and/or an anti-lock braking system (ABS) and/or an electronic stability program (ESP). Additionally or alternatively, the detection device can be designed to save the data about the at least one transportation vehicle occupant detected with regard to the driving maneuver or to collect the data and to determine and/or store a statistical pattern in the behavior or activity of the transportation vehicle occupant on the basis of the data.

Disclosed embodiments also include a transportation vehicle having the disclosed system. The transportation vehicle may be configured as an automobile, especially a passenger car.

Disclosed embodiments also include developments of the system and the transportation vehicle, which comprise the features that have already been described in connection with the developments of the disclosed method. For this reason, the corresponding developments of the disclosed system and the disclosed transportation vehicle will not be described again here.

In the exemplary embodiment, the described components of the embodiment each represent individual features that should be considered independently of each other and that also each develop independently of each other and that should thus also be considered integral parts, individually or in a different combination from the one shown. Furthermore, the described embodiment can also be supplemented by more of the previously described features.

In a first method operation at S1, a transportation vehicle occupant 12 in an interior 14 of the transportation vehicle 16 is detected by a detection device 10 of a system for adapting at least one operating parameter of a transportation vehicle 16. The transportation vehicle occupant 12 can be a driver or a passenger of the transportation vehicle 16, for example. When detecting the transportation vehicle occupant 12, the detection device 10 can additionally or alternatively be configured to identify the detected transportation vehicle occupant 12. To identify the transportation vehicle occupant 12, the detection device 10 can be configured, for example, to detect biometric features, such as the body size and/or face and/or voice and/or weight of the transportation vehicle occupant 12. To detect the face and/or the body size of the transportation vehicle occupant 12, the detection device 10 can have a camera, for example. To detect the weight of the transportation vehicle occupant 12, the detection device 10 can have a weight sensor, for example. To detect the voice of the transportation vehicle occupant 12, the detection device 10 can have a microphone, for example, which is configured to perform voice recognition. To identify the transportation vehicle occupant, the control system can be designed to compare the detected data relating to the transportation vehicle occupant with data stored in the control system. In addition, the control system can be configured to associate the data with a transportation vehicle occupant. As soon as the transportation vehicle occupant 12 has been detected and/or identified by the detection device 10, the control system can be configured to specify transportation vehicle parameters and/or operating parameters that have been pre-set for the transportation vehicle occupant 12 or to retrieve them from a transportation vehicle-external unit. In other words, pre-set transportation vehicle parameters and/or operating parameters can be assigned to at least one driver assistance system or multiple driver assistance systems of the transportation vehicle 16 specifically for the transportation vehicle occupant who has been detected. If the transportation vehicle 16 drives away or is put into operation, then the transportation vehicle 16 or a driving maneuver by the transportation vehicle 16 is controlled as a function of the pre-set parameters or parameter sets.

In operating the transportation vehicle 16, the at least one operating parameter or the transportation vehicle parameters of the transportation vehicle 16 can be pre-set. For example, a driving profile can be associated with the identified transportation vehicle occupant 12. The at least one operating parameter of the transportation vehicle 16 or multiple operating parameters of the transportation vehicle 16 can be saved in the driving profile. The driving profile or multiple driving profiles, each of which can be assigned to a transportation vehicle occupant, can be stored in a memory device of the control system (not shown in the figure), for example. The control system can be configured to associate the detected transportation vehicle occupant 12 with the driving profile. The at least one operating parameter of the transportation vehicle 16 or multiple operating parameters of the transportation vehicle 16 can be pre-set by the driving profile that has been determined. For example, the predetermined distance maintained by the adaptive cruise control can be saved for the detected transportation vehicle occupant 12.

The transportation vehicle 16 may be configured as an automobile, especially a passenger car. Moreover, the transportation vehicle 16 has at least one driver assistance system. The transportation vehicle may have multiple driver assistance systems. The at least one driver assistance system is configured to perform or control or monitor a driving maneuver of the transportation vehicle 16. An operating parameter of the transportation vehicle 16 is set by the at least one driver assistance system of the transportation vehicle 16 during the driving maneuver. In other words, the driver assistance system of the transportation vehicle 16 can set or provide at least one operating parameter of the transportation vehicle 16 for the performance of the driving maneuver.

For example, the driving maneuver can be the driving of the transportation vehicle 16 and/or passing another transportation vehicle and/or changing lanes and/or turning a corner and/or driving backward and/or stopping and/or starting and/or accelerating and/or driving through curves and/or pulling the transportation vehicle 16 into a parking space and/or pulling out of a parking space. The at least one driver assistance system or multiple driver assistance systems are responsible for performing the respective driving maneuver. To do so, the respective driver assistance system sets at least one operating parameter of the transportation vehicle 16. The at least one operating parameter can be a velocity of the transportation vehicle and/or an acceleration, especially a lateral acceleration, of the transportation vehicle and/or a longitudinal acceleration of the transportation vehicle and/or a distance from a transportation vehicle driving ahead and/or the keeping of the transportation vehicle in a driving lane and/or a steering torque.

For example, to perform the driving maneuver of driving the transportation vehicle 16 through a curve, the at least one driver assistance system can provide a steering torque and/or lateral acceleration as the at least one operating parameter. If the at least one driver assistance system is an adaptive cruise control, for example, then the driver assistance system is configured to maintain a predetermined distance from a transportation vehicle traveling ahead at a predetermined velocity of the transportation vehicle 16 as a driving maneuver and/or to initiate a braking procedure if the distance falls below the predetermined distance from the transportation vehicle traveling ahead. With the adaptive cruise control, the operating parameter can be provided as the predetermined distance from a transportation vehicle traveling ahead and/or a velocity of the transportation vehicle 16.

In a second method operation at S2, an activity by the transportation vehicle occupant 12 during the driving maneuver in reaction to the driving maneuver is detected by the detection device 10. In other words, the detection device 10 can be designed to detect a change in a state of the transportation vehicle occupant 12. At this juncture, the detection device 10 can be designed to recognize a conscious behavior and/or unconscious behavior of the transportation vehicle occupant 12. By recognizing the conscious and/or unconscious behavior of the transportation vehicle occupant 12, conclusions can be drawn about the current driving maneuver. The activity by the transportation vehicle occupant 12 that the detection device 10 is configured to detect can be an action H by the transportation vehicle occupant 12 and/or a state parameter Z of the transportation vehicle occupant 12. For example, while the transportation vehicle 16 is accelerating or driving through a curve as the current driving maneuver, the detection device 10 can be configured to detect, for instance, the approach of a transportation vehicle occupant's foot toward a brake pedal or grabbing a hand grip of the transportation vehicle 16 as actions by the transportation vehicle occupant 12.

By detecting the action H by the transportation vehicle occupant 12, a control system can, in a third method operation at S3, adapt the at least one operating parameter of the transportation vehicle 16 in response to the detected activity by the transportation vehicle occupant 12 by activating the at least one driver assistance system. If the at least one driver assistance system specifies an acceleration as the at least one operating parameter while the transportation vehicle 16 is driving through the curve as the current driving maneuver of the transportation vehicle 16, then the control system can, for instance, reduce the acceleration or speed when adapting the at least one operating parameter as a result of detecting the action H. For example, if the detection device 10 detects the approach of a foot toward an accelerator pedal of the transportation vehicle 16 as the action H by the transportation vehicle occupant 12 while the transportation vehicle is driving through the curve as the driving maneuver, then the control system can, for instance, increase the acceleration when adapting the at least one operating parameter.

Hereafter, four more concrete exemplary embodiments will be described.

In the first exemplary embodiment, a driver gets into the transportation vehicle 16 as the transportation vehicle occupant 12 of the transportation vehicle 16 for the first time. The detection device recognizes biometric data or other data relating to the driver in the interior 14 of the transportation vehicle 16. Additionally or alternatively, the detection device 10 can be configured to compare the detected biometric data or other data with further data, which are stored in a transportation vehicle-external unit. In this exemplary embodiment, the detection device 10 can determine that no data about a desired driving mode of the transportation vehicle occupant 12 are available yet for the transportation vehicle occupant 12. Thus generally determined, pre-set operating parameters or parameter values can optionally be used, or the driver is asked to provide desired parameters. A sporty or defensive driving mode can be set as the driving mode, for example. In a first driving situation, the detection device 10 recognizes that the driver himself or herself would have braked somewhat earlier due to the reflexive gripping of a door handle by the driver, for example, when a second transportation vehicle merges in front of his or her own transportation vehicle, and it initiates braking. In response, the distance parameter as the operating parameter is modified in the driver assistance system to make the driver feel better when a similar situation occurs again. This parameter modification can be communicated to the transportation vehicle-external central computer or system, if appropriate, so that the parameters can likewise be adapted for other first-time drivers of the transportation vehicle 16.

In the second exemplary embodiment, the driver gets into the transportation vehicle 16 as the transportation vehicle occupant 12. A determination is made by the detection device 10, e.g., sensors, that the driver's heart rate is elevated. Additionally, it is detected by the detection device that an important appointment is saved in the appointment calendar and can barely be reached on time. The system assumes that the driver is in a hurry and therefore, for this drive, sets the maximum lateral acceleration as an operating parameter for negotiating curves.

In the third exemplary embodiment, the driver gets into the transportation vehicle 16 and places a child seat in the back seat of the transportation vehicle 16, which is recognized by the detection device 10. In response, the control system of the system may be configured to specify or set a defensive driving mode, i.e., a more defensive choice of parameters, independently.

In the fourth exemplary embodiment, the detection device 10 recognizes that the driver would like to drive faster on the way to work in the morning than on the way back in the evening. In response, the control system adapts the parameters of the driver assistance system or the multiple driver assistance systems to this time of day-based rhythm.

Overall, the disclosed embodiments show how the passenger comfort zone is automatically recognized.

### LIST OF REFERENCE SIGNS

10 Detection device
12 Transportation vehicle occupant
14 Interior
16 Transportation vehicle
H Action
S1 First method operation
S2 Second method operation
S3 Third method operation
Z State parameter

1. A transportation vehicle with a system for adapting at least one operating parameter of the transportation vehicle during a driving maneuver of the transportation vehicle, the system comprising:
at least one driver assistance system configured to set the at least one operating parameter, wherein, a detection device is configured to detect at least one activity by a transportation vehicle occupant during the driving maneuver in reaction to the driving maneuver; and
a control system configured to adapt the at least one operating parameter of the transportation vehicle in response to the detected activity by the transportation vehicle occupant by activating the at least one driver assistance system.

2. The transportation vehicle of claim 1, wherein the heart rate and/or the respiratory rate and/or the brain activity and/or the drowsiness of at least one transportation vehicle occupant is detected as the activity.

3. The transportation vehicle of claim 2, wherein a predetermined action by at least one transportation vehicle occupant that indicates an intervention by the transportation vehicle occupant in the driving maneuver is detected as the activity, wherein a foot movement by the transportation vehicle occupant that indicates an actuation of a brake pedal or an accelerator pedal of the transportation vehicle is detected as the predetermined action, or the predetermined action indicates an actuation of a steering wheel of the transportation vehicle.

4. The transportation vehicle of claim 1, wherein a protective action by at least one transportation vehicle occupant for the personal protection of the transportation vehicle occupant is detected as the activity, wherein a vocal utterance by the transportation vehicle occupant and/or holding tightly and/or bracing by the transportation vehicle occupant against an interior element of the transportation vehicle is detected as a protective action.

5. The transportation vehicle of claim 1, wherein a velocity and/or a lateral acceleration and/or a longitudinal acceleration and/or a distance from a transportation vehicle driving ahead and/or the keeping of the transportation vehicle in a driving lane and/or a steering torque is adapted as the at least one operating parameter.

6. The transportation vehicle of claim 1, wherein the body size and/or face and/or voice and/or weight of the transportation vehicle occupant is detected for the identification of the transportation vehicle occupant by biometric features.

7. The transportation vehicle of claim 1, wherein the system detects a confirmation action to a status query produced by an output device with respect to the reaction to the driving maneuver by the transportation vehicle occupant to adapt the at least one operating parameter of the transportation vehicle.

8. The transportation vehicle of claim 1, wherein the detection device detects data from a device that include appointment entries and/or personal calendar data, which are considered when the at least one operating parameter is adapted.

9. A system for adapting at least one operating parameter of the transportation vehicle during a driving maneuver of the transportation vehicle, the system comprising:
at least one driver assistance system configured to set the at least one operating parameter, wherein a detection device is configured to detect at least one activity by a transportation vehicle occupant during the driving maneuver in reaction to the driving maneuver; and
a control system configured to adapt the at least one operating parameter of the transportation vehicle in response to the detected activity by the transportation vehicle occupant by activating the at least one driver assistance system.

10. The system of claim 9, wherein the detection device includes a camera and/or a sensor.

11. The system of claim 9, wherein the heart rate and/or the respiratory rate and/or the brain activity and/or the drowsiness of at least one transportation vehicle occupant is detected as the activity.

12. The system of claim 9, wherein a predetermined action by at least one transportation vehicle occupant that indicates an intervention by the transportation vehicle occupant in the driving maneuver is detected as the activity, wherein a foot movement by the transportation vehicle occupant that indicates an actuation of a brake pedal or an accelerator pedal of the transportation vehicle is detected as the predetermined action, or the predetermined action indicates an actuation of a steering wheel of the transportation vehicle.

13. The system of claim 9, wherein a protective action by at least one transportation vehicle occupant for the personal protection of the transportation vehicle occupant is detected as the activity, wherein a vocal utterance by the transportation vehicle occupant and/or holding tightly and/or bracing by the transportation vehicle occupant against an interior element of the transportation vehicle is detected as a protective action.

14. The system of claim 9, wherein a velocity and/or a lateral acceleration and/or a longitudinal acceleration and/or a distance from a transportation vehicle driving ahead and/or the keeping of the transportation vehicle in a driving lane and/or a steering torque is adapted as the at least one operating parameter.

15. The system of claim 9, wherein the body size and/or face and/or voice and/or weight of the transportation vehicle occupant is detected for the identification of the transportation vehicle occupant by biometric features.

16. The system of claim 9, wherein the system detects a confirmation action to a status query produced by an output device with respect to the reaction to the driving maneuver by the transportation vehicle occupant to adapt the at least one operating parameter of the transportation vehicle.

17. The system of claim 9, wherein the detection device detects data from a device that include appointment entries and/or personal calendar data, which are considered when the at least one operating parameter is adapted.

18. A method for adapting at least one operating parameter of a transportation vehicle during a driving maneuver of the transportation vehicle, wherein the at least one operating parameter of the transportation vehicle is set by at least one driver assistance system of the transportation vehicle, the method comprising:
identifying at least one transportation vehicle occupant, wherein biometric features are detected during the identification of the transportation vehicle occupant;
establishing or loading a predetermined driving profile for the transportation vehicle occupant;
detecting at least one activity by a transportation vehicle occupant during the driving maneuver in response to the driving maneuver by a detection device;
adapting the at least one operating parameter of the transportation vehicle in response to the detected activity by the transportation vehicle occupant by activating the at least one driver assistance system using a control system; and
storing the at least one adapted operating parameter in the driving profile of the transportation vehicle occupant.

19. The method of claim 18, wherein the heart rate and/or the respiratory rate and/or the brain activity and/or the drowsiness of at least one transportation vehicle occupant is detected as the activity.

20. The method of claim 18, wherein a predetermined action by at least one transportation vehicle occupant that indicates an intervention by the transportation vehicle occupant in the driving maneuver is detected as the activity, wherein a foot movement by the transportation vehicle occupant that indicates an actuation of a brake pedal or an accelerator pedal of the transportation vehicle is detected as the predetermined action, or the predetermined action indicates an actuation of a steering wheel of the transportation vehicle.

21. The method of claim 18, wherein a protective action by at least one transportation vehicle occupant for the personal protection of the transportation vehicle occupant is detected as the activity, wherein a vocal utterance by the transportation vehicle occupant and/or holding tightly and/or bracing by the transportation vehicle occupant against an interior element of the transportation vehicle is detected as a protective action.

22. The method of claim 18, wherein a velocity and/or a lateral acceleration and/or a longitudinal acceleration and/or a distance from a transportation vehicle driving ahead and/or the keeping of the transportation vehicle in a driving lane and/or a steering torque is adapted as the at least one operating parameter.

23. The method of claim 18, wherein the body size and/or face and/or voice and/or weight of the transportation vehicle occupant is detected for the identification of the transportation vehicle occupant by biometric features.

24. The method of claim 18, wherein the system detects a confirmation action to a status query produced by an output device with respect to the reaction to the driving maneuver by the transportation vehicle occupant to adapt the at least one operating parameter of the transportation vehicle.

25. The method of claim 18, wherein the detection device detects data from a device that include appointment entries and/or personal calendar data, which are considered when the at least one operating parameter is adapted.
